(19) United States
(12) Patent Application Publication
(10) Pub. No.: US 2004/0191780 A1
(43) Pub. Date: Sep. 30, 2004

(54) Simple and efficient tissue culture process of producing viable plants of Decalepis hamiltonii using vescicular arbuscular mycorrhizae (VAM)

(76) Inventors: Giridhar Parvatam, Mysore (IN); Thammannan Rajasekaran, Mysore (IN); Gokare Aswathanarayana Ravishankar, Mysore (IN)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/402,241

(22) Filed: Mar. 31, 2003

(51) Int. Cl. C12Q 1/68

(52) U.S. Cl. 435/6

(57) ABSTRACT

A simple and efficient method for producing viable plants by tissue culture from a Decalepis hamiltonii nodal explants and their effective field establishment by using vesicular arbuscular mycorrhizae (VAM) for effective growth and desired yield of flavor enhanced tubers; three culture media first medium, second medium, third medium to achieve the same, and also, a method of altering the level of flavor in plant Decalepis hamiltonii, said method comprising the tissue culturing the stem explants of various species of plant Decalepis hamiltonii.

### FIELD OF THE PRESENT INVENTION

A simple and efficient method for producing viable plants by tissue culture from a Decalepis hamiltonii nodal explants and their effective field establishment by using vesicular arbuscular mycorrhizae (VAM) for effective growth and desired yield of flavour enhanced tubers; three culture media first medium, second medium, third medium to achieve the same, and also, a method of altering the level of flavour in plant Decalepis hamiltonii, said method comprising the tissue culturing the stem explants of various species of plant Decalepis hamiltonii.

### BACKGROUND AND PRIOR ART REFERENCES

Decalepis hamiltonii Wight & Arn., (swallow root) belonging to Asclepiadaceae is a monogeneric climbing shrub native of the Deccan peninsula and endemic to the forest areas of Western Ghats of India. It finds use as a culinary spice due to its high priced aromatic roots. These are also used in herbal medicines. The present invention deals with a tissue culture process for the development of a large number of plants from a specified part of Decalepis hamiltonii plant. The process of the present invention opens up new possibilities for producing highly efficient Decalepis hamiltonii tubers with enhanced flavour content and also for hairy root cultures for secondary metabolites by using modem techniques of agrobiotechnology.

The roots are markedly fleshy, cylindrical (1-6 cm diameter) are characterized by a sarasaparilla like taste accompanied by a tingling sensation on the tongue as described in Wealth of India 1952 (Wealth of India 1952, A dictionary of raw materials, CSIR, New Delhi 3: 24). The roots of D.hamiltonii are used as a flavouring principle (Wealth of India, 1990), appetizer (Murthi, P. B. R., and Seshadri,T. R. Proc. Ind.Acad.Sci. 1947; 13A, 221), blood purifier (Jacob, K. C. Madras Agric. Journal. An unrecorded economic product Decalepis hamiltonii W & Arn., Family Asclepidaceae 1937; 25; 176), and preservative (Phadke, N. Y., Gholap A. S., Ramakrishnan K, Subbulakshmi G., JFood Sci. Technol. 1994; 31, 472). Similarly the roots of this taxon as described by Nayar et al. (1978) (Nayar R C, Shetty J K P, Mary Z and Yoganrasimhan 1978.

Pharmacological studies of root of Decalepis hamiltonii W & Arn and comparison with Hemidesmus indicus (L.) R.Br. Proc. Indian Acad. Sciences 87 (B): 37-48) are considered as “Sariva Bheda” in Ayurveda where finds use as an alternative to roots of Hemidesmus indicus in the preparation of several herbal drugs like Amrutamalaka taila, Drakshadi chuma, shatavari rasayana and yeshtimadhu taila The main objective of the present invention is to provide a simple process for large scale tissue culture based micropropagation of Decalepis hamiltonii. Another objective of the present invention is to provide a powerful tool for the isolation of flavour content 2-hydroxy-4-methoxy benzaldehyde of yielded tubers.

Plant regeneration by tissue culture techniques is well established. A wide variety of plant species has been successfully regenerated in vitro via organogenesis or somatic embryogenesis. Organogenesis leads to organ formation i.e. shoot (or root), which can be isolated to induce development of roots (or shoots) to produce full plants while somatic embryogenesis leads to the development of somatic embryos (embryos developed without genetic fertilization) which have both shoot and root initially and are capable of developing into whole plants. Although the ability of individual parts of plants and cells to regenerate into complete plants (called totipotency) is a well known phenomenon, each plant or plant part requires specialised studies to invent the conditions that allow such regeneration. Some of the factors controlling growth and differentiation of such cultures have been determined.

The establishment of interactions among different groups of phytohormones, and growth regulators alone or in combinations are responsible for certain interrelations existing among cells, tissues and organs. So there seems to be consensus that the success in inducing differentiation depends upon the type of plant part (“explant”), the physiological condition of the explant and physical and chemical milieu of explant during culture. Due to this, the science of tissue culture has been directed to optimize the physiological conditions of source plant, the type of explant, the culture conditions and the phytohormones used to initiate tissue culture. This substantiates the fact that development of a new process for proliferation of plants by tissue culture is not obvious.

One major aspect that has to be investigated on case-by-case basis is the type of plant growth regulators and the amount of plant growth regulators that induce regeneration. Besides, chemical composition of the medium, temperature and other culture conditions play an important role in the induction of organogenesis and somatic embryogenesis and their maturation to healthy fertile plants thereof. The response to medium, hormones and growth conditions differs from plant species to species and variety to variety. Thus inventing conditions for efficient regeneration of plants, requires developing specialized knowledge about a given plant.

Another major area where innovativeness is required in tissue culture, is identifying the plant part that efficiently responds to the culture conditions and leads to prolific regeneration. Not all plant parts of a given species are amenable to efficient regeneration. It is a complex combination of the explant selected identified for regeneration, physiological state of the explant, growth conditions and growth regulators that determines success of a plant in tissue culture. Different explants from a given plant usually show entirely different and often unpredictable response to growth conditions for proliferation. No general principles can be applied to achieve regeneration. In each case, identification of the explant and identification of the culture conditions are innovative steps in the development of a tissue culture method for regeneration of a plant part into a number of plants. In fact, conditions are determined after much experimentation. The Applicants have prepared many experiments and after much trial and error were ble to arrive at the plant parts and the ingredients used in the process, and various parameters involved in the steps of the process.

Yet another important aspect in Micropropagation of plants is the hardening and successful field transfer of tissue cultured plants. Considerable progress has been made over the years, in furthering knowledge on various dimensions of Mycorrhiza, especially vesicular arbuscular mycorrhizae (VAM). Due to their beneficial and stimulating effects on plant growth, meeting nutritive deficiency of zinc, phosphorous and nitrogen as bio-fertilizers (Mukherjji and Chamola 1997,) in soils of arid and semi arid tropical; countries, induced suppression of soil/root borne fungi and resistance of water stress etc., their exploration in soils of different agroclimatic zones has been taken up (Rathi 1992). Due to high cost of fertilizers and with a view to maintain the ecosystem of soil, addition of fertilizer has to be minimized which is done by adding biofertilizer in soil. Among various microbial inoculants, VAM is one which stimulates plant growth in soils of low fertility providing phosphate to plants (Christopher et al 1994).

Till this date, very few reports are available for regeneration of Decalepis hamiltonii through tissue culture. But the processes described earlier are not very efficient. The starting materials (explant) used in the earlier processes were different. For example, these processes shoot buds, axillary buds and leaf pieces as the starting material. In this respect scanty reports on tissue culture of Decalepis hamiltonii have been published. Even profuse callusing from the base of the explant which normally hinders growth of the shoots is another drawback. Some of these are also related to the establishment of callus, and are listed below for convenience and reference.

Application of tissue culture techniques for the production and biosynthesis of useful plant constituents has been exploited for the production of secondary metabolites from excised root culture, callus and by crown gall tissue in a number of plants. (West F R. Jr and Mike E S 1957. Synthesis of atropine by isolated roots and root callus cultures of belladona, Botan.Gaz. 119:50-54; Klein R M 1960, Plant tissue culture: a possible source of plant constituents, Econ. Botany 14: 286-289). For example cell suspension and callus cultures of Mentha piperita & M. spicata were reported to enable the production and biosynthesis of secondary metabolites (Lin and Staba 1961, Peppermint and spearmint tissue cultures, callus formation and submerged culture, Leoydia 24:139-145; Wang and Staba 1963, Peppermint and spearmint Tissue culture II: Dual-Carboy culture of spearmint Tissue. Jour of Pharmaceutical Science 52:1058-1062).

Such cell suspensions were later reported to biotransform certain precursors into monoterpenes (Aviv D and Gulan E 1978. Biotransformation of monoterpenes by Mentha cell lines: Conversion of pulegone to isomenthone. Planta Medica 33; 70-77;). Of late the highly aromatic roots have been subjected to over exploitation by destructive harvesting that has endangered the survival of this plant. In the earlier reports by George et al. (George, J. Perira, J., Divakar, S., Udayasankar, K and Ravishankar, G. A. Current Science,1999; 77, 501-502) it was observed that the aromatic roots of D.hamiltonii proved to be a potent bioinsecticide on storage pests at lethal and sub-lethal levels (Indian Patent No. 1301/Del/98). The supercritical extracts of these roots proved to be potent antimicrobial agents (George J., Udayasankar, K., Keshava, N and Ravishankar, G. A. Fitoterapia 1999; 70, 172-174).

Harsh Pal Bais, Jacob George, and Ravishankar, G. A. (Current Science,2000; 79:894-898) were able to regenerate plantlets of D.hamiltonii W&A from leaf callus. Similarly a method for rooting of Decalepis hamiltonii for field transfer was reported earlier (Bais H P, Sudha G, Suresh B &. Ravishankar G A, Curr. Sci, 2000, 79: 408-410; Obul Reddy, B., Giridhar, P and Ravishankar G. A, Current Science 81(11), 2001,1479-1482). These reports deal with the multiplication of shoots from pre-existing meristems in axis of leaves, and up to 5-6 shoots could be obtained from single explant of Decalepis hamiltonii. Apart from this the leaf based protocols, however, are not efficiently reproducible and produce only a few shoots per explant. Although differentiation of shoots from callus was observed, the efficiency was extremely low with only 40-50% response.

TABLE 1
Summarizes the state of art tissue culture processes related to
Decalepis plant as covered by patents or described in literature.
It is then followed by statement describing the process invented
by us in contrast to the known state of art.
State of art of tissue culture work on Decalepis

| | | |
|---|---|---|
| | Mode of | |
| | Regeneration | |
| | Phyto- | |
| | hormones | |
| Report | Explant | Remarks |

1. Harsh Pal Bais, Jacob George and G. A. Ravishankar 2000

| | | |
|---|---|---|
| In vitro propaga- | Clonal | Clonal propagation of |
| tion of Decalepis | propagation | Decalepis hamiltonii |
| hamiltonii Wight & | BAP, NAA | by using axillary bud |
| Arn an endangered | Axillary | cultures wre reported. |
| shrub through | buds | The influence of BAP |
| axillary bud | | and NAA combination |
| cultures. | | was studied. But pro- |
| Current Scinece. | | fuse callusing from |
| 79: 408-410. | | the base of explants |
| | | is a draw back which |
| | | hinders further growth |
| | | of shoot and root |
| | | formation. |

2. Jacob Geroge, Harsh Pal Bais, G. A. Ravishankar 2000

| | | |
|---|---|---|
| Optimization of | callus | In this report response |
| media constituents | BAP, NAA | surface methodology was |
| for shoot regener- | leaf | utilized in statistical |
| ation from leaf | | optimization of three |
| callus cultures of | | quality facotrs such as |
| Decalepis hamiltonii | | the number of shoots, |
| Wight & Arn., Hort | | shoot length and number |
| Science 35, | | of leaves, pertaining |
| 296-299. | | to regeneration of |
| | | plantlets from leaf |
| | | callus of Decalepis |
| | | hamiltonii. The variable |
| | | evaluated were the |
| | | levels of sucrose, BAP |
| | | and NAA. Reproduc- |
| | | ibility of this |
| | | protocol is very low. |

3. Harsh Pal Bais, G. Sudha, B. Suresh and G. A. Ravishankar 2000

| | | |
|---|---|---|
| Silver nitrate | In Vitro | Effects of silver ni- |
| influences in vitro | Rooting | trate on in vitro |
| root formation in | IAA | rooting of tissue |
| Decalepis hamiltonii | In vitro | cultured shoots were |
| Wight & Arn. Current | shoots | described. The combi- |
| Science 79, | | nation of silver nitrate |
| 894-898. | | and IAA on in vitro |
| | | rooting was highlighted |
| | | and also ethephon. The |
| | | influence of other |
| | | auxins was not studied. |

4. B. Obul Reddy, P. Giridhar and G. A. Ravishankar 2001

| | | |
|---|---|---|
| In vitro rooting of | In vitro | In this report the sig- |
| Decalepis hamiltonii | rooting | nificance of different |
| Wight and Arn an | IAA, IBA, | root promoting agents |
| endangered shrub | NAA | such as phloroglucinol, |
| by auxins and | In vitro | cobalt chloride, silver |
| root promoting | shoots | nitrate and activated |
| agents. Current | | charcoal along with |
| Science | | auxins IAA, IBA and |
| | | NAA were reported. But |
| | | this study was confined |
| | | to in vitro rooting |
| | | only. |

5. B. Obul Reddy, P. Giridhar and G. A. Ravishankar 2001

| | | |
|---|---|---|
| The effect of | shoot multi- | Describes the effect of |
| triacontanol on | plication | triacontanol, NAA and |
| micropropagation of | IAA, IBA, | BAP in medium on the |
| Capsicum frutescens | NAA | in vitro multiple shoot |
| and Decalepis | In vitro | formation and in vitro |
| hamiltonii Wight & | shoots | rooting of Decalepis |
| Arn., Plant Cell | | hamiltonii. But number |
| Tissue and Organ | | of shoots formed per |
| Culture. | | explant was less than |
| 71: 253-258. | | six. |

Novelties in the Present Invention Vis a Vis State of Art

The present invention provides an efficient tissue culture process for producing viable plants, improvement of their growth and yield of flavour enhanced tubers of Decalepis hamiltonii by using Vescicular arbuscular mycorrhizae for giving a large number of mature plants. This is potentially very useful in plant biotechnology for micropropagation, selecting variants and genetic transformation. The process of this invention is very simple and is applicable to commercial cultivation of the Decalepis hamiltonii. The process also provides a simple method to alter the composition of flavor component of tubers.

The process of the present invention employs the nodal region from two months old brach of 2 Y old green house grown plants (for obtaining fully developed plants) as a starting material (explant), which is slightly different from all the earlier reports (as given in Table 1) wherein, either in vitro nodal explants or explants of unknown age were used (not mentioned). The process of the present invention for inducing a high frequency of organogenesis leads to whole plant development where the de novo regenerants are from tissues other than preexisting meristems. We could identify an explant that when cultured in suitable medium in the presence of certain combinations of commonly used growth regulators can stimulate a high frequency of differentiation of shoots. Unlike reports 2 and 5 in Table 1, our process gives a larger number of shoots. Report 3 in Table 1 gives particularly poor regeneration from Decalepis hamiltonii which is not the case with our process. Unlike reports 4 and 11 in Table 1, the nodal explant used by us is very convenient to obtain multiple shoots.

Earlier art dealing with multiple shoot formation used either shoot tips or nodal tissue as the explant which consists of preexisting meristematic tissues in the form of axillary buds or shoot tips. The pre-existing meristematic tissue in such explants, when cultured in the presence of growth regulators starts growing to give a few shoots. The present invention also uses nodal explant of two months old branches that does contain preexisting primordia cultured in the hormonal concentrations used along with modified vitamin composition. The nodal explant gives a large number of shoots. This nodal segment of two months old branches has not been used in any earlier report for the regeneration of plants.

The phytohormone combinations and the explants used in the present invention are quite different from those used in any of the reports described in Table 1. The multiple shoot regeneration in our protocol was successful within certain limits of the phytohormone levels. For example, 2iP (gamma..gamma. dimethyl allyl amino purine) functions efficiently at concentration of 4.92 .mu.M to 13.7 .mu.M with indole acetic acid at 0.57 .mu.M to 5.71 .mu.M. But BAP works at 4.44 .mu.M to 11.1 .mu.M with indole acetic acid 0.57 .mu.M to 5.71 .mu.M moderately. As described in Table 1 these ranges and combinations of phytohormone have not been used earlier for the development of a process for multiple shoot regeneration in Decalepis hamiltonii plants

### OBJECTS OF THE PRESENT INVENTION

Therefore the main object of the present invention is to provide a simple and reproducible tissue culture process for regeneration of a large number of Decalepis hamiltonii plants from their explants (node) which obviates the drawbacks in the processes reported earlier as detailed above.

Another object of the present invention is to provide an improved growth and yield of flavour enhanced tubers of Decalepis hamiltonii which provides better economic value.

### SUMMARY OF THE PRESENT INVENTION

A simple and efficient method for producing viable plants by tissue culture from a Decalepis hamiltonii nodal explants and their effective field establishment by using vesicular arbuscular mycorrhizae (VAM) for effective growth and desired yield of flavour enhanced tubers; three culture media first medium, second medium, third medium to achieve the same, and also, a method of altering the level of flavour in plant Decalepis hamiltonii, said method comprising the tissue culturing the stem explants of various species of plant Decalepis hamiltonii.

### DETAILED DESCRIPTION OF THE PRESENT INVENTION

A simple and efficient method for producing viable plants by tissue culture from a Decalepis hamiltonii nodal explants and their effective field establishment by using vesicular arbuscular mycorrhizae (VAM) for effective growth and desired yield of flavour enhanced tubers; three culture media first medium, second medium, third medium to achieve the same, and also, a method of altering the level of flavour in plant Decalepis hamiltonii, said method comprising the tissue culturing the stem explants of various species of plant Decalepis hamiltonii.

In an embodiment of the present invention, wherein a simple and efficient method for producing viable plants by tissue culture from a Decalepis hamiltonii nodal explants and their effective field establishment by using vesicular arbuscular mycorrhizae (VAM) for effective growth and desired yield of flavour enhanced tubers, said method comprising the steps of:

Cutting an explant from a Decalepis hamiltonii plant, said explant consisting essentially of the nodal segment containing axillary bud of said Decalepis plant,

Decontaminating said explant by removing from its surface any contaminant which is potentially harmful to the tissue culture process,

Culturing the decontaminate (ii) explant at a temperature between 25 and 30 degree C., in the presence of cool white light in a first medium which is capable of producing multiple shoots, said first medium having a pH in the range of 5.4 to 6.0 being sterile as a result of autoclaving and comprising:

salts

vitamins

a carbon source

phytohormones comprising auxins and cytokinins in a concentration of greater than 4.0 micro molar and

a gelling agent

continuing the culture of said explant until proliferating shoots are formed,

culturing said shoots in a second medium which is capable of further elongation of shoot at temperature between 25 and 30 degree C. in the presence of a cool white light for at least 4 weeks to generate 6-8 cm long shoots, said second medium having a pH in the range of 5.4 to 6.0, being sterile as a result of autoclaving and comprising:

salts

vitamins

a carbon source

phytohormones

a gelling agent

Culturing said elongated shoots in a third medium which is capable of inducing roots, at a temperature between 25 and 30 degree C., in the presence of a cool white light for at least 4 weeks to induce rooting, said third medium having a pH in the range of 5.4 to 6.0, being sterile as a result of autoclaving and comprising:

Salts

vitamins

a carbon source

phytohormones

a gelling agent

Hardening the rooted plants by removing carefully from the third medium and washing the medium under running tap water and their subsequent planting in the micropots containing a sand-compost mixture (1:2) under the polythene hoods in the green house for 4 weeks,

Development of seedling based plantlets by sowing the fresh seeds of D. hamilltonii in garden soil. Separation of 12-15 cm long seedlings and planting in pots, inoculation of VAM to the seedling plants and also to hardened micropropagated plants planted in pots comprising:

VAM inoculum

Mixture of soil : red earth: farm yard manure

Growth of the plants for six months in green house with a photoperiod of 16:8 hourrs relative humidity of 70 to 78 percent during light cycle and 80 to 86 percent during darkness,

Measurement of vegetative growth and yield of tubers of the said VAM treated Decalepis hamiltonii platnelts,

Slicing of the said harvested tubers of Decalepis hamiltonii and analysis of the flavor component 2-hydroxy 4 methoxy benzaldehyde by known method using GC.

In another embodiment of the present invention, wherein said first medium, said second medium and said third medium comprise, salts and modified vitamins of Murashige and Skoog medium

In yet another embodiment of the present invention, wherein said nodal segment from a Decalepis plant grown in the field is treated to remove any contaminant

In still another embodiment of the present invention, wherein said first medium, said second medium and said third medium comprise salts of Murashige and Skoog medium

In still another embodiment of the present invention, wherein said first medium, said second medium, and said third medium comprise the following salts of Murashige and Skoog medium:

| Component | Concentration (mg/L) |
|---|---|
| Salts of Murashige and Skoog medium: | |
| NH.sub.4 NO.sub.3 | 1650 |
| KNO.sub.3 | 1900 |
| MgSO.sub.4 7H.sub.2 O | 180.54 |
| MnSO.sub.4 H.sub.2 O | 16.90 |
| ZnSO.sub.4 7H.sub.2 O | 8.6 |
| CuSO.sub.4 5H.sub.2 O | 0.025 |
| CaCl.sub.2 H.sub.2 O | 332.02 |
| KI | 83 |
| CoCl.sub.2 2H.sub.2 O | 0.025 |
| KH.sub.2 PO.sub.4 | 170 |
| H.sub.3 BO.sub.3 | 6.20 |
| Na.sub.2 MoO.sub.4 2H.sub.2 O | 0.25 |
| Fe Na. EDTA | 36.70 |
| Myoinositol | 100.0 |

In still another embodiment of the present invention, wherein the concentration of said salts of Murashige and Skoog medium is at the full level on weight by volume basis.

In still another embodiment of the present invention, wherein said vitamins of said first medium, said second medium, and said third medium comprise:

| Component Concentration | (mg/L) |
|---|---|
| Nicotinic acid | 0.5 |
| Pyridoxine HCl | 0.5 |
| Thiamine HCl | 0.1 |
| Glycine | 2.0 |
| Biotin | 0.05 |

In still another embodiment of the present invention, wherein said carbon source in first medium, said second medium, and said third medium is selected from the group consisting of sucrose and glucose.

In still another embodiment of the present invention, wherein said carbon source in said first medium, second medium and third medium is at a range of 2-4 percent w/v.

In still another embodiment of the present invention, wherein said first medium further comprises or cytokinin or combination thereof.

In still another embodiment of the present invention, wherein the cytokinin is selected from the group consisting of 6-benzylaminopurine (BAP), kinetin and gamma.gamma.dimethyl allyl amino purine (2iP) at a concentration range varying between 4-15 mu.M.

In still another embodiment of the present invention, wherein the phytohormones in said second medium are selected from the group consisting of cytokinins, auxins and combinations thereof.

In still another embodiment of the present invention, wherein the auxin in said first medium and said second medium is selected from the group consisting of auxins, indole aetic acid, indole butyric acid and naphthalene acetic acid at a concentration in the range of 0.05 to 10 mu.M.

In still another embodiment of the present invention, wherein the phytohormones in said first medium and said second medium are cytokinins selected from the group consisting of 6-benzylaminopurine (BAP), kinetin and gamma.gamma.dimethylallyl amino purine (2iP) at a concentration range varying between 4-15 mu.M.

In still another embodiment of the present invention, wherein the cytokinin in said first medium is selected from the group consisting of 6-benzylaminopurine (BAP), kinetin and gamma.gamma.dimethylallyl amino purine (2iP) at a concentration range varying between 4-15 mu.M.

In still another embodiment of the present invention, wherein the phytohormone in said second medium is an auxin selected from the group consisting of auxins indole aetic acid, indole butyric acid and naphthalene acetic acid at a concentration in the range of 0.05 to 10 mu.M.

In still another embodiment of the present invention, wherein said auxin in third medium is selected from the group consisting of auxins indole aetic acid, indole butyric acid and naphthalene acetic acid at a concentration in the range of 0.05 to 10 mu.M.

In still another embodiment of the present invention, wherein the explant is decontaminated by dipping in a solution containing at least one sterilizing agent.

In still another embodiment of the present invention, wherein said sterilizing agent is selected from the group consisting of sodium hypochlorite, mercuric chloride and ethyl alcohol.

In still another embodiment of the present invention, wherein the gelling agent is selected from the group consisting of agar and gelrite at a concentration range 0.2 to 0.8% w/v.

In still another embodiment of the present invention, wherein said shoots can be used for micropropagation of Decalepis plants.

In still another embodiment of the present invention, wherein the VAM inoculum is added to the soil mixture in pots containing plants in the range of 35 to 70 gm per pot (5700 cc volume soil per pot)

In still another embodiment of the present invention, wherein the VAM treated plants of Decalepis hamiltonii showed better growth and yield of tubers.

In still another embodiment of the present invention, wherein the tubers of VAM treated plants of Decalepis hamiltonii showed altered levels of 2 hydroxy 4 methoxy benzaldehyde depending upon the VAM strain used in soil.

In still another embodiment of the present invention, wherein the Decalepis plant tubers with altered levels of flavor content useful for industrial applications.

In still another embodiment of the present invention, wherein the tissue cultured Decalepis hamiltonii plants are useful for effective commercial propagation.

In still another embodiment of the present invention, wherein the shoot length increases by about 4.5 times.

In still another embodiment of the present invention, wherein the number of nodes increases by about 1.6 times.

In still another embodiment of the present invention, wherein the number of leaves increases by about 1.6 times.

In still another embodiment of the present invention, wherein the total chlorophyll content increases by about 80%.

In still another embodiment of the present invention, wherein the total number of tubers increases by tuber diameter increases by about 1.6 times.

In still another embodiment of the present invention, wherein the tuber length increases by about one time.

In still another embodiment of the present invention, wherein fresh weight of tuber increases by about 1.2 times.

In still another embodiment of the present invention, wherein the flavour content increases about 4.6 times.

In still another embodiment of the present invention, wherein a first medium for the efficient production of viable plants by tissue culture from a Decalepis hamiltonii nodal explants and their effective field establishment by using vesicular arbuscular mycorrhizae (VAM) for effective growth and yield of flavour enhanced tubers, said first medium comprising:

| Component | Concentration (mg/L) |
|---|---|
| Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100 |
| B. Vitamins | |
| Nicotinic acid | 0.5 |
| Pyridoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Cytokinins | 4 to 15 .mu.M |
| Auxins | 0.05 to 10 .mu.M |
| E. Gelling Agents | 0.2 to 0.8% w/v |

In still another embodiment of the present invention, wherein a second medium for the efficient production of viable plants by tissue culture from a Decalepis hamiltonii nodal explants and their effective field establishment by using vesicular arbuscular mycorrhizae (VAM) for effective growth and yield of flavour enhanced tubers, said first medium comprising:

| Component | Concentration (mg/L) |
|---|---|
| Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100 |
| B. Vitamins | |
| Nicotinic acid | 0.5 |
| Pyridoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Cytokinins | 4 to 15 .mu.M |
| Auxins | 0.05 to 10 .mu.M |
| Gibberellins | 0.2 to 0.4 .mu.M |
| E. Gelling Agents | 0.2 to 0.8% w/v |

In still another embodiment of the present invention, wherein third medium for the efficient production of viable plants by tissue culture from a Decalepis hamiltonii nodal explants and their effective field establishment by using vesicular arbuscular mycorrhizae (VAM) for effective growth and yield of flavour enhanced tubers, said first medium comprising:

| Component | Concentration (mg/L) |
|---|---|
| Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100.0 |
| B. Vitamins | |
| Nicotinic acid | 0.5 |
| Pyndoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Auxins | 0.05 to 10 .mu.M |
| E. Gelling Agents | 0.2 to 0.8% w/v |

In still another embodiment of the present invention, wherein a method of altering the level of flavour in plant Decalepis hamiltonii, said method comprising the tissue culturing the stem explants by the above-stated method.

The present invention relates to a tissue culture process for producing a large number of viable plants of D. hamiltonii, their growth improvement and yield of 2 hydroxy 4 methoxy benzaldehyde in tubers. The process of the present invention employs specified pieces of nodal segments of the stem of D. hamiltonii as the staring material and identifies medium and culture conditions for producing a large number of plants. Such plants can be used for micropropagation. The process of the present invention also employs application of vesicular arbuscular mycorrhizae for effective establishment of micropropagated plants and also growth and yield of flavor enhanced tubers of D. hamiltonii.

The present invention relates to a tissue culture process for producing a large number of viable Decalepis hamiltonii plants in vitro. The process of the present invention employs specified pieces of stem (nodal explants) of the Decalepis plant as the starting material and identifies media and culture conditions for producing a large number of plants. Such plants can be used for micropropagation and also for commercial propagation of Decalepis hamiltonii.

To meet the above objects, the applicants now provide a method of regenerating a large number of viable and fertile Decalepis hamiltonii plants by tissue culture technique starting from a small tissue(explant) of Decalepis hamiltonii plant, said method comprising:

i) cutting the nodal segment (explant) of Decalepis hamiltonii plants,

ii) removing any contaminants such as fungus, bacteria, microbes etc. which are potentially harmful to the process, from the surface of the nodal segments (explants),

iii) culturing the decontaminated nodal segments from step (ii) in first medium capable of producing an shoots, said first medium consisting of:

a) Salts of any conventional medium

b) Vitamins of any conventional medium,

c) Carbon source,

d) Phytohormones (plant growth regulators), and

e) Gelling agent

at a pH in the range of 5.4 to 6.0 and sterilizing the medium by autoclaving. The culturing was effected at the temperature 20-30.degree. C. in the presence of cool white light

iv) continuing the culture of the said nodal segments until proliferating shoots are formed,

v) Further culturing of the shoots obtained from step (iv) on second medium capable of elongation and further growth and harvesting the shoots formed, said second medium comprising:

Salts of any conventional medium

b) Vitamins of any conventional medium,

c) Carbon source,

d) Phytohormones (plant growth regulators),

e) Gibberellin (GA3) and

f) Gelling agent.

at a pH in the range of 5.4 to 6.0 and sterilizing the medium by autoclaving the culturing was effected at the temperature 20-30.degree. C. in the presence of cool white light for a minimum period of four weeks for elongation and further growth of shoots.

vi) culturing the shoots obtained from step (v) in third medium capable of inducing roots, said third medium comprising:

a) Salts of any conventional medium

b) Vitamins of any conventional medium,

c) Carbon source,

d) Phytohormones (plant growth regulators), and

e) Gelling agent

at a pH in the range of 5.4 to 6.0 and sterilizing the medium by autoclaving the culturing was effected at the temperature 20-30.degree. C. in the presence of cool white light for a minimum period of two weeks to generate roots.

In the present invention the nodal segments employed are those obtained from two months old branch of two years old plants grown in the field or those grown by the tissue culture in the laboratory. The node used from the Decalepis hamiltonii plants grown in the field are treated by conventional methods to remove the contaminants.

The first, second and third medium employed in the invention comprise salts and modified vitamins of MS medium, carbon source and gelling agent. The preferred Murashige and Skoog (MS) medium comprise the following salts:

| Component | Concentration (mg/L) |
|---|---|
| (a) Salts of Murashige and Skoog medium: | |
| NH.sub.4 NO.sub.3 | 1650 |
| KNO.sub.3 | 1900 |
| MgSO.sub.4 7H.sub.2 O | 180.54 |
| MnSO.sub.4 H.sub.2 O | 16.90 |
| ZnSO.sub.4 7H.sub.2 O | 8.6 |
| CuSO.sub.4 5H.sub.2 O | 0.025 |
| CaCl.sub.2 H.sub.2 O | 332.02 |
| KI | 83 |
| CoCl.sub.2 2H.sub.2 O | 0.025 |
| KH.sub.2 PO.sub.4 | 170 |
| H.sub.3 BO.sub.3 | 6.2 |
| Na.sub.2 MoO.sub.4 2H.sub.2 O | 0.25 |
| Fe Na. EDTA | 36.70 |
| Myoinositol | 100.0 |

Further, the preferred vitamins used in the first, second and third medium are:

| Component | Concentration (mg/L) |
|---|---|
| Nicotinic acid | 0.5 |
| Pyridoxine HCl | 0.5 |
| Thiamine HCl | 0.1 |
| Glycine | 2.0 |
| Biotin | 0.05 |

In addition, the preferred carbon source used in the first and the second medium is selected from sucrose or glucose and is employed at a range of 2 to 4% w/v.

The phytohormones employed in the first medium are selected from cytokinins, or auxins or a combination thereof. More specifically, the auxin employed is selected from the group consisting of indole acetic acid, indole butyric acid, and naphthalene acetic acid at a concentration range varying between 0.05 to 10 .mu.M, and the cytokinins employed in the first medium is selected from a group consisting of 6-benzylaminopurine, .gamma..gamma. dimethyl allyl aminopurine and kinetin at a concentration range varying between 4 to 15 .mu.M.

On the other hand, the preferred phytohormones employed in the third medium are selected from auxins such as indole acetic acid, indole butyric acid and naphthalene acetic acid at a concentration of up to 10 .mu.M.

The decontamination of the explant is effected by dipping in a solution containing at least one sterilizing agent selected from the group consisting of sodium hypochlorite, calcium hypochlorite, mercuric chloride, ethyl alcohol etc.

The gelling agent used is selected from agar, gelrite (phytagel) or any gelling agent at a concentration range 0.2 to 0.8% w/v.

The concentration of salts of the MS medium mentioned in steps (iii) and (vi) was used in full quantities mentioned above on weight by volume basis. The shoots obtained by the said tissue culture process can be used for micropropagation of Dealepis hamiltonii plants.

The different VAM cultures used in this study Glomus mosseae, G. fasciculatum, and G. monosporum efficiently improved the vegetative growth and tubers yield of hardened plants of both tissue cultured and seedling plants.

The regenerated shoots contain altered/unaltered levels of secondary metabolites depending on phytohormone combinations used in the medium. The VAM treated plants in the present invention contain altered/unaltered levels of flavour content 2 hydroxy 4 methoxy benzaldehyde useful for industrial application. The micro shoots can be used for genetic transformation based on infection by Agrobacterium or via bombardment of DNA coated microparticles.

The most preferred process of the present invention comprises:

i) cutting the nodal segment (explant) of Decalepis hamiltonii plants,

ii) removing any contaminants such as fungus, bacteria, microbes etc which are potentially harmful to the process, from the surface of the nodal segments (explants),

iii) culturing the decontaminated nodal segments from step (ii) in a medium given in Table 2.

TABLE 2

| Component | Concentration (mg/L) |
|---|---|
| A. Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100 |
| B. Vitamins | |
| Nicotinic acid | 0.5 |
| Pyridoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Cytokinins | 4 to 15 .mu.M |
| Auxins | 0.05 to 10 .mu.M |
| E. Gelling Agents | 0.2 to 0.8% w/v |

at a pH in the range of 5.4 to 6.0, sterilizing the medium by autoclaving, and the culturing being effected at a temperature in the range of 20-30.degree. C. in the presence of cool white light,

iv) continuing the culture of said nodal segments until proliferating shoots are formed,

v) harvesting the shoots formed,

vi) Further culturing the shoots from step (v) in a medium given in table.3

TABLE 3

| Component | Concentration (mg/L) |
|---|---|
| A. Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100 |
| B. Vitamins | |
| Nicotinic acid | 0.5 |
| Pyridoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Cytokinins | 4 to 15 .mu.M |
| Auxins | 0.05 to 10. mu.M |
| Gibberellins | 0.2 to 0.4 .mu.M |
| E. Gelling Agents | 0.2 to 0.8% w/v |

vii) culturing the shoots in a medium employed for the formation of roots as given in Table 4.

TABLE 4

| Component | Concentration (mg/L) |
|---|---|
| A. Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100.0 |
| B. Vitamins | |
| Nicotinic acid | 0.5 |
| Pyndoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Auxins | 0.05 to 10 .mu.M |
| E. Gelling Agents | 0.2 to 0.8% w/v |

at a pH in the range of 5.4 to 6.0 and sterilizing the medium by autoclaving, effecting the culturing at a temperature in the range of 20-30 .degree. C. The plantlets so formed, if desired, according to requirements, can be transferred to the soil for growing Decalepis hamiltonii plants on a very large scale.

Two month old seedlings with shoot length of approximately 12-15 cm were used for studying the effect of VAM. Three different strains of VAM fungi viz. Glomus mosseae, Glomus fasciculatum, Glomus mosnosporum were used as a inoculum. The starter inoculum of each VAM strain was prepared by multiplying in sterile pots containing sterile soil by sowing the ragi seeds. After 4 weeks the seedlings that emerged were taken out carefully and checked under microscope for % of VAM infection to roots and counted the number of spores in roots and per gram of soil.

Both green house raised seedlings and micropropagated plants can be selected by providing VAM treatment of their effective field survival and better growth. The VAM treatments consisted of Glomus mosseae, Glomus fascicultum, Glomus mosnosporum and uninoculated (controls). The VAM inoculum can be made easily by any known method by using ragi seeds or mustard seeds or any other suitable host of the strains of VAM selected for this purpose.

After one month growth the % infection of the roots of the host plant can be calculated if necessary, otherwise the segments of the host plant root system can be used directly in the required proportion. Fill the pots containing plants with mixture of soil: red earth: farm yard manure in the range of 2:1:1 (5700 cc soil mixture per pot). Inoculation of VAM was done at the rate of 50 g pot⁻¹ (soil along with root pieces containing 15-16 spores per gram of soil) at the depth of 5 cm. After 3 months of growth biometric observations like per cent root infection, plant height, number of nodes, number of leaves, number of tubers, size of tubers, fresh weight of tubers and the flavour content of tubers along with chlorophyll content of leaves was recorded. Later the tubers were separated from plant and washed in water remove the adhering soil particles.

According to another feature of the invention, the node segments employed may be those obtained from the plants grown in the fields or those grown by tissue culture in the laboratory. Particularly in the case of nodes used from the plants grown in the fields, it is essential to treat them to remove the contaminants. This treatment can be made by any conventional methods which include treatment with hypochlorites, mercuric chloride, ethyl alcohol etc.

The hormones (growth regulators) employed in the culture medium may be selected from cytokinins such as BAP(6 benzyl amino purine or 6-benzyl adenine), 2iP (.gamma., .gamma. dimethyl allylamino purine), kinetin; auxins such as IAA (indole acetic acid), NAA (naphthalene acetic acid), IBA (indole butyric acid). Gelling agents such as agar 0.6 to 0.8 % w/v or gelrite (phytagel) 0.2 to 0.5% or any gelling agent at suitable concentration may be employed for the generation of organogenic callus and for proliferation of shoots.

The concentration of the salts of the MS medium (the component mentioned at A in the tables 2 & 3) may be the full quantities mentioned in the Tables or at half the level on weight by volume basis.

We have found that by culturing the basal differentiating mass or cuttings from the newly formed shoots using steps (iii) to (iv) it is possible to proliferate more shoots and obtain large number of healthy Decalepis hamiltonii plants repeatedly.

According to one aspect of this invention, multiple shoots can be isolated repeatedly from the primary shoots of the cultured explant after the first cycle of this, invention.

According to another aspect of this invention, the shoots obtained from primary shoots can be further elongated to produce adventitious shoots.

According to yet another aspect of this invention, plantlets obtained from explants can be rooted and such rooted plants can be shifted to soil and grown normally.

According to still another embodiment the method of this invention can be employed for successful establishment of the rooted plants by employing VAM in the soil which leads to higher vegetative growth rate and improve the yield of tubers and altered levels of flavour content of tubers which are economically important.

The process of the present invention is described in details below:

To get the nodal segments, the plant material may be collected from the field grown Decalepis hamiltonii plants or shoot cultures maintained in a tissue culture laboratory. Nodal segments of size 1.5 cm or smaller may be collected for use. The nodal segments collected from tissue culture raised plants maintained in the laboratory can be used directly for culture by the process of the present invention to obtain multiple shoots, while the nodal segments collected from field grown plants are first treated for removing contaminants such as bacteria or fungus which are potentially harmful to the process of the present invention.

To ensure that the explant is free of bacteria and fungi infections (contaminants) in the medium, the explant is surface sterilized before use. Many sterilizing techniques are available in the art for the purpose of preparing explant for culture. Such techniques involve dipping the explant in the solution containing at least one sterilizing agent. Such sterilizing agents include, sodium hypochlorite, calcium hypochlorite, mercuric chloride, ethyl alcohol etc. Here the explant can be surface sterilized by dipping the explant in 1-2 % sodium hypochlorite solution for 5-15 min. with continued shaking, followed by washing thoroughly with excess of deionized sterile water (5-6 times).

The surface sterilized explant (nodal segments), can be placed aseptically for culturing. The medium may consist of Murashige and Skoog (MS) salts,and vitamins at full concentration as given in component A of the tables on weight by volume basis or any other conventional medium or any other vitamin composition known in the art, carbon source of sucrose or glucose at 2 to 4% w/v, and growth regulators of sufficient concentration to induce callus and shoot formation. Growth regulators may be selected from cytokinins such as 6-benzyl amino purine, kinetin, .gamma..gamma. dimethyl allylamino purine etc.; auxins such as indole acetic acid, indole butyric acid, napthalene acetic acid. Gelling agent may be for e.g. agar 0.6 to 0.8% or gelrite (phytagel) 0.2 to 0.5% w/v.

The pH of the medium may be adjusted to 5.4 to 6.0 prior to autoclaving. Up to 10 explants can be placed in each of 300 ml Magenta vessels containing 50 ml medium or single explant can be cultured in 50 ml glass tubes containing 15 ml culture medium. The cultures may be incubated at temperature 20-30 degree. C. in light (at least 40 .mu.mol/m.sup.2 s) 16 h photoperiod. The light can be provided from white fluorescent tubes or any other source of cool white light. The culture of the explant may be continued till several shoots are formed on the original explant. The distinct and well formed proliferating shoots may be harvested.

The shoots can be harvested in sterile environment (laminar flow) with the help of a sharp scalpel and blade. The harvested shoots can be transferred to another medium which promotes induction and growth of roots. The rooting medium may contain Murashige and Skoog salts at full strength or any other conventional medium and vitamins of Murashige and Skoog or any other known vitamin composition, sucrose or glucose 2 to 4% w/v; commonly used auxin type growth regulators in the art for this purpose e.g. indole acetic acid, napthalene acetic acid, indole butyric upto 10 .mu.M concentration; gelling agent e.g. agar 0.6 to 0.8% w/v or gelrite 0.2 to 0.5% w/v pH 5.6-6.0 prior to autoclaving. The culture may be incubated at the temperature 20-30.degree. C. in light (40 .mu.mol/m.sup.2 s) 16 h photoperiod. Culturing may be continued till well developed roots are formed.

The shoots with well developed root system can be taken out of the culture, roots can be washed thoroughly with excess of water to remove traces of agar and nutrients from the surface of roots. The plantlets can now be transferred to micropots containing soil mixture containing sand and farm yard manure (1:1) covered with polythene covers and should grow under green house conditions for hardening for about 4 weeks and later can be transplanted to field.

The well established plants in micropots can be used for field transfer and VAM inoculum can be introduced into the pit or rhizosphere zone of plant for its efficient acclimatization and improved growth and yield of the tubers.

The process of the present invention for inducing multiple shoots, their in vitro rooting and hardening leads to whole plant development. We could identify an explant that, when cultured in suitable medium in the presence of certain combinations of commonly used growth regulators, can stimulate a high frequency of differentiation of regenerants and the technique can be used effectively. Under the given culture conditions the explant of Decalepis hamiltonii is subjected to revised programming of cells resulting in the production of large number of shoots. The significant aspect of using growth regulators is the they facilitate clonal propagation of the Decalepis hamiltonii.

Earlier art dealing with multiplying shoot formation used shoot tips as the explant which consists of pre-existing meristematic tissues. The pre-existing meristematic tissue in such explants, when cultured in the presence of growth regulators starts growing to give a single or few shoots. The present invention uses two months old nodal explant that too contain pre-existing primordia and the nodal explant gives a large number of shoots when cultured in medium described in the process, that to be with out any basal callusing, unlike very few shoots along with basal callusing in both shoot formation and rooting stage as reported earlier.

The following examples are given by way of illustration of the present invention and should not be constructed to limit the scope of the present invention.

### EXAMPLE 1

Multiple Shoot Formation in Decalepis hamiltonii Wight & Arn., (Swallow Root) Swallow root (Decalepis hamiltonii) is an economically important endangered shrub since it contains a very high content of 2 hydroxy 4 methoxy benzaldehyde an aromatic flavour compound in its tubers as a major flavour compound along with many medicinal properties. So far, tissue culture method for efficient regeneration is not available for this swallow root plant. Here, we describe the applicability of the process according to present invention for Decalepis hamiltonii.

Nodal segments(explant) were cut from the field grown Decalepis hamiltonii. Nodal segments were treated to remove bacteria/fungus (contaminants) by dipping the segments in 1% sodium hypochlorite for 10 min with continued shaking. The explants were then washed thoroughly with excess of deionised sterile water (5-6 times) and trimmed at the cut ends. The decontaminated nodal segments were placed in medium consisting of Murashige and Skoog salts, and vitamins, sucrose 3% w/v, growth regulator 2iP at 9.94 .mu.M concentration in combination with indole acetic acid at 0.57 .mu.M concentration, gelling agent agar (0.7%) or phytagel (0.2%) w/v. The pH was adjusted at 5.8 prior to autoclaving at 121.degree.0 C., 15 lb/inch.sup.2 for 20 min.

The explants were placed on the medium with the help of sterile forceps in laminar flow. Cultures were incubated at 25.+−.2.degree. C. in light (40 .mu.mol/m.sup.2 s) 16 h photoperiod. Culturing continued till shoots initiating out of it. Initiation of shoots occurred within four weeks time with a frequency of 80-90%. In the absence of cytokinin type growth regulators or in their presence at a low concentration (below 9.94 .mu.M), differentiation of shoots from explant could not occur. However, on medium containing 2iP (dimethyl allylamino purine) (9.94 .mu.M) along with IAA (0.57 mu.M.) several shoots (8 to 9) were initiated in six weeks time in culture. Again at higher concentration of 2iP (13.76 mu.M) along with IAA (0.57 mu.M.) very few shoots were produced. For harvesting the shoots, the cultures were taken out of the culture vessels and shoots were cut with the help of a sharp scalpel blade in a laminar flow.

The shoots were again cultured on shoot elongation medium containing Murashige and Skoog salts and vitamins, sucrose 3% w/v, auxin type growth regulator indole acetic acid (0.57 .mu.M), BAP (8.88 mu.M.), GA₃ (0.29 mu.M.) and gelling agent agar 0.6% w/v. The pH was adjusted to 5.8 prior to autoclaving at 121.degree. C., 15 lb/cm.sup.2 for 20 min. For promoting formation of roots, the cultures were incubated in the above medium at 25.+−.2.degree. C. in light (40 .mu.mol/m.sup.2 s) 16 hr. photoperiod. Culturing was continued for 4 weeks for further development and also for the development of more adventitious shoots.

Shoots were then separated aseptically under laminar flow and transferred to a culture medium containing Murashige and Skoog salts and vitamins, sucrose 3% w/v, auxin type growth regulator indole butyric acid (7.36 .mu.M), and gelling agent agar 0.6% w/v. The pH was adjusted to 5.8 prior to autoclaving at 121.degree. C., 15 lb/inch.sup.2 for 20 min. For promoting formation of roots, the cultures were incubated in the above medium at 25.+−.2.degree. C. in light (40 .mu.mol/m.sup.2 s) 16 hr. photoperiod. Culturing was continued till roots were formed. Well developed root system was formed within 3 weeks time when the plantlets were ready to transfer into soil. The plants were acclimatized for autotrophic growth, prior to transfer in soil.

### EXAMPLE 2

Inoculation of VAM into Pots Containing Decalepis hamiltonii Plants

Two month old seedlings with shoot length of approximately 12-15 cm were used for studying the effect of VAM. Similarly the same length microproapagated and hardened plants also can be used. Three different strains of VAM fungi viz. Glomus mosseae, Glomus fasciculatum, Glomus mosnosporum were used as a inoculum. The starter inoculum of each VAM strain was prepared by multiplying in sterile pots containing sterile soil by sowing the ragi seeds. After 4 weeks the seedlings that emerged were taken out carefully and checked under microscope for % of VAM infection to roots and counted the number of spores in roots and per gram of soil.

The treatments consisted of T1) Glomus mosseae, T2) Glomus fasciculatum, T3) Glomus mosnosporum and T4) uninoculated (controls). Pots were filled with mixture of soil: red earth: farm yard manure in the range of 2:1:1 (5700 cc soil mixture per pot). Inoculation of VAM was done at the rate of 50 g pot⁻¹ (soil along with root pieces containing 15-20 spores per gram of soil) at the depth of 5 cm. After 3 months of growth biometric observations like per cent root infection, plant height, number of nodes, number of leaves, number of tubers, size of tubers, fresh weight of tubers and the flavour content of tubers along with chlorophyll content of leaves was recorded.

The results revealed that, in case of seedling plants, among the VAM species used Glomus mosseae found to be the most efficient in colonizing the roots, and improved the bioemtric characters like plant height (72.2±1.78 cm), number of nodes (13.4±0.89), number of leaves(26.2±1.78), number of tubers (10.6±1.51), fresh weight of tubers (16±0.72) compared to G. fasciculatum and G. monosporum and controls. Glomus mosseae treated plant tubers showed high content of chlorophyll (Table. 1). The tubers were separated from plant and washed in water to remove the adhering soil particles.

### EXAMPLE 3

Isolation and Analysis of the Flavour Compound 2-hydroxy 4 methoxy benzaldehyde in Harvested Tubers

Then the washed tubers were mechanically dissected into small pieces of 0.5-1.0 cm diameter, and subjected to steam distillation for 5 hours. The steam condense was extracted with dichloromethane (50 ml×4). The combined extracts were passed through a funnel containing anhydrous sodium sulphate to remove the water content, concentrated in a flash evaporator and dissolved in 1 ml ethanol and stored in closed vials.

Quantification of the flavour compound was determined by gas chromatographic analysis (GC) using flame ionization detection (FID)

Analysis of 2-hydroxy-4-methoxybenzaldehyde (2H4MB) was done by spotting the root extracts on TLC plate along with standard (Fluka Chemicals, Switzerland) and run in a solvent system comprising of Hexane: Benzene (1:1). Rf of spot coinciding with that of standard (2H4MB) (0.47) was eluted in solvent and UV spectrum was measured on a Perken-Elmer UV-V is recording spectrophotometer UV-160. Maximum absorption was obtained at 278 nm. Quantitative detection was done by GC(FID). The constituent was identified by matching the mass spectra with GC-MS library user generated mass spectral libraries, and also confirmed by comparison with GC retention time of standard sample.

The concentrated volatiles were separated by GC, flame ionization detector (FID) with capillary column and GC-MS analysis using a Shimadzu, GC-14B coupled with QP 5000 MS system under the following conditions SPB-1 column (Supelco, USA, 30 m×0.32 mm, 0.25 μM film thickness); oven temperature programme, 60° C. for 2 min, rising at 2° C./min to 250° C., held for 5 min; injection port temperature 225° C.; detector temperature, 250° C.; carrier gas helium, flow rate 1 ml min⁻¹. The amount of solution injected was 1 ml for analysis. The GC(FID) profiles indicated that there was marginal improvement in the flavour content (2-hydroxy-4-methopxy benzaldehyde)in tubers of both treated and control plants (Table 5 & 6).

Effect of Vescicular Arbuscular Mycorrhizae Inoculation on Growth and Yield of Decalepis hamiltonii Seedling Plants as Shown in Table.5

| Parameter | Control | G. mosseae | G. fasciculatum | G. monosporum |
|---|---|---|---|---|
| Shoot length | 14.2 ± 1.46 | 72.2 ± 2.86 | 30 ± 3.16 | 25.95 ± 1.5 |
| (cm) | | | | |
| Number of | 5.16 ± 0.40 | 13.4 ± 0.89 | 10.4 ± 1.01 | 8.4 ± 5.16 |
| nodes | | | | |
| Number of | 10.4 ± 0.89 | 26.2 ± 1.78 | 20.2 ± 1.88 | 16.8 ± 2.22 |
| leaves | | | | |
| Total | 13.88 ± 1.60 | 24.18 ± 1.26 | 24.14 ± 1.32 | 19.19 ± 0.98 |
| chlorophyll (mg | | | | |
| g⁻¹ FW) | | | | |
| Number of | 4.2 ± 0.45 | 10.6 ± 1.51 | 6.2 ± 0.84 | 6.0 ± 0.70 |
| tubers | | | | |
| Range of tuber | 0.5-0.9 | 1.0-2.5 | 1.0-1.4 | 0.5-1.4 |
| diameter (cm) | | | | |
| Range of tuber | 0.5-0.9 | 2.0-8.2 | 1.2-5.8 | 2.0-6.2 |
| length (cm) | | | | |
| Fresh weight of | 9.59 ± 0.55 | 16.0 ± 0.72 | 13.3 ± 0.80 | 10.4 ± 0.63 |
| tubers (gm) | | | | |
| Flavour content | 0.0006 | 0.003 | 0.002 | 0.0009 |
| (2H4MB)(%) | | | | |

Data recorded after 3 months of growth of two months old seedlings given VAM treatment. Results are an average of 5 samples (mean ± S.D.)
*Amount of VAM inoculum used 50 gm l⁻¹

Effect of Vescicular Arbuscular Mycorrhizae Inoculation on Growth and Yield of Micropropagated Plants of Decalepis hamiltonii as Shown in Table.6

| Parameter | Control | G. mosseae | G. fasciculatum | G. monosporum |
|---|---|---|---|---|
| Shoot length | 15.5 ± 0.96 | 80.5 ± 1.50 | 36.7 ± 0.58 | 33.3 ± 0.56 |
| (cm) | | | | |
| Number of | 6.0 ± 0.85 | 15.0 ± 0.50 | 11.0 ± 0.45 | 9.2 ± 0.85 |
| nodes | | | | |
| Number of | 12 ± 0.95 | 30.0 ± 0.65 | 22.0 ± 0.85 | 18.0 ± 0.38 |
| leaves | | | | |
| Total | 14.2 ± 0.86 | 24.5 ± 1.85 | 24.2 ± 0.52 | 21.6 ± 0.58 |
| chlorophyll (mg | | | | |
| g⁻¹ FW) | | | | |
| Number of | 4.5 ± 0.50 | 11.5 ± 0.85 | 6.5 ± 0.98 | 6.2 ± 0.55 |
| tubers | | | | |
| Range of tuber | 0.5-0.9 | 1.0-2.8 | 1.0-1.6 | 0.5-1.4 |
| diameter (cm) | | | | |
| Range of tuber | 0.5-0.9 | 2.0-9.4 | 1.4-6.5 | 2.0-6.5 |
| length (cm) | | | | |
| Fresh weight of | 9.85 ± 0.95 | 18.65 ± 0.85 | 14.5 ± 0.35 | 12.6 ± 0.46 |
| tubers (gm) | | | | |
| Flavour content | 0.0008 | 0.0045 | 0.0028 | 0.001 |
| (2H4MB) (%) | | | | |

Data recorded after 3 months of growth of two months old seedlings given VAM treatment. Results are an average of 5 samples (mean ± S.D.)
*Amount of VAM inoculum used 50 gm l⁻¹

So the Decalepis hamiltonii plants can be developed by clonal propagation and the improvement of the vegetative growth and yield of tubers with containing altered/unaltered levels of flavour metabolite 2 hydroxy 4 methoxy benzaldehyde useful in cuisine and pharmaceutical applications.

In accordance with the various aspects of this invention, an easy, efficient and rapid method is provided for inducing shoots at high frequency. The process of this invention provides differentiation and offers many advantages over the prior art, which are obtained out of human interference and totally unobvious. Indeed, the results/inferences of this process are surprising and the inventors themselves could no believe that they would be able to achieve such an enhanced results. The reproducibility and rapidity clonal propagation and the chance in the level of flavour metabolites obtainable routinely by this process is expected to facilitate genetic transformation of Decalepis hamiltonii via Agrobacterium and/or biolistic based transformation techniques. An additional advantage of this invention is that only one explant gives several shoots within one or subsequent step. Mass propagation as well as selection of mutants can now be expedited with the application of this invention.

### EXAMPLE 4

In order to see the efficiency of the VAM fungi used in this study on other plant systems an experiment was conducted wherein, the VAM treatment was given to micro propagated Vanilla planifolia plantlets. The micro propagated Vanilla plantlets were produced in the laboratory by the method that standardized earlier (Giridhar, P., Obul Reddy B and Ravishankar G. A. Silver nitrate influences in vitro shoot multiplication and root formation in Vanilla planifolia Current Science, 2001, 81: 101-103.). Plantlets with shoot length of approximately 11-15 cm were used for studying the effect of VAM. Three different strains of VAM fungi viz. Glomus mosseae, Glomus fasciculatum, Glomus mosnosporum were used as a inoculum. The starter inoculum of each VAM strain was prepared by multiplying in sterile pots containing sterile soil by sowing the ragi seeds. After 4 weeks the seedlings that emerged were taken out carefully and checked under microscope for % of VAM infection to roots and counted the number of spores in roots and per gram of soil.

The treatments consisted of T1) Glomus mosseae, T2) Glomus fasciculatum, T3) Glomus mosnosporum and T4) uninoculated (controls). Pots were filled with mixture of soil: red earth: farm yard manure in the range of 2:1:1 (5700 cc soil mixture per pot). Inoculation of VAM was done at the rate of 50 g pot⁻¹ (soil along with root pieces containing 15-20 spores per gram of soil) at the depth of 5 cm. After 3 months of growth biometric observations like per cent root infection, plant height, number of nodes, number of leaves, along with chlorophyll content of leaves was recorded. The results revealed that, among the VAM species used none of them were found to be effective in showing any impact on the vegetative growth of this Vanilla plant. Even Glomus mosseae found to be poor in colonizing the roots and it didn't showed any improvement on the growth of the plant compared to control plants (Plants not given VAM treatment). The control plants were with a shoot length of 20-22 cm and 6-7 nodes (Data was not given as there was no improvement in treated plants compared to controls). In general the degree of association of VAM with roots is related to the plant inherent factors such as physiology, metabolism and plant growth rate (Warner A and Mosse B, Independent spread of vesicular-arbuscular myorrhizal fungi in soil. Trans. Br. Mycol. Soc., 1990, 74:407-410.) From this study it is clear that the improvement of growth of a plant by using VAM varies with the plant system.

So the Decalepis hamiltonii plants can be developed by clonal propagation and the improvement of the vegetative growth and yield of tubers with containing altered/unaltered levels of flavour metabolite 2 hydroxy 4 methoxy benzaldehyde useful in cuisine and pharmaceutical applications.

In accordance with the various aspects of this invention, an easy, efficient and rapid method is provided for inducing shoots at high frequency. The process of this invention provides differentiation and offers many advantages over the prior art. The reproducibility and rapidity clonal propagation and the chance in the level of flavour metabolites obtainable routinely by this process is expected to facilitate genetic transformation of Decalepis hamiltonii via Agrobacterium and/or biolistic based transformation techniques. An additional advantage of this invention is that only one explant gives several shoots within one or subsequent step. Mass propagation as well as selection of mutants can now be expedited with the application of this invention.

Here again, the applicants found to their surprise that VAM is not only effective but imparted tremendous growth to the plant and also, provided other advantages. Hence, the invention is totally novel and inventive.

References

1. Wealth of India, “A dictionary of raw materials”, CSIR, New Delhi 3: 24 (1952)

2. P. B. R. Murthi and Seshadri, T. R. Proc. Ind.Acad.Sci.; 13A, 221(1947)

3. K. C Jacob, “An unrecorded economic product Decalepis hamiltonii W & Arn., Family Asclepidaceae”, Madras Agric. Journal. 25: 176(1937)

4. N. Y. Phadke, A. S. Gholap, K. Ramakrishnan and G. Subbulakshmi, “Essential oil of Decalepis hamiltonii as an antimicrobial agent”, J.Food Sci.Technol. 31: 472 (1994)

5. R. C. Nayar, J. K. P. Shetty, Z. Mary and Yoganrasimhan. “Pharmacological studies of root of Decalepis hamiltonii W & Arn and comparison with Hemidesmus indicus (L.) R.Br.” Proc. Indian Acad. Sciences 87 (B): 37-48(1978)

6. K. G. Mukherjii. and B. P. Chamola,“Vescicular arbuscular mycorrhizal fungi in afforestation.” In National Conference on Plant Biotechnology, Bareilly (ed. S. K. Bhatnagar) pp.1, (1997)

7. S. K. Rathi,. “Studies on mycorrhizae of some cultivated and wild plants in Meerut,” Ph.D. thesis. C.C.S. University, Meerut., U. P., India. (1992)

8. C. Christopher, G. Rangaraju and P. K. Krishna. “Importance of VAM”. Intensive Agriculture, 25: 23-25 (1994)

9. F. R. West. Jr and E. S. Mike, “Synthesis of atropine by isolated roots and root callus cultures of belladona”, Botan.Gaz. 119:50-54(1957).

10. R. M. Klein, “Plant tissue culture: a possible source of plant constituents”, Econ. Botany 14: 286-289(1960).

11. Lin and Staba, Peppermint and spearmint tissue cultures, callus formation and submerged culture, Leoydia 24:139-145(1961)

12. Wang and Staba, Peppermint and spearmint Tissue culture II: Dual-Carboy culture of spearmint Tissue. Jour of Pharmaceutical Science 52:1058-1062(1963)

13. D. Aviv and E. Gulan. Biotransformation of monoterpenes by Mentha cell lines: Conversion of pulegone to isomenthone. Planta Medica 33: 70-77 (1978).

14. J. George, J. Perira, S. Divakar, K. Udayasankar and G. A Ravishankar,“Bioinsecticide from swallow root (Decalepis hamiltonii Wight & Arn) protects food grains against insect infestation”, Current Science, 77: 501-502 (1999)

15. J. George, K. Udayasankar, N. Keshava, and G. A. Ravishankar, “Antimicrobial activity of supercritical extract from Decalepis hamiltonii roots”, Fitoterapia 1999; 70, 172-174 (1999).

16. Harsh Pal Bais, Jacob George, and G. A. Ravishankar, “In vitro propagation of Decalepis hamiltonii Wight & Arn an endangered shrub through axillary bud cultures”. Current Science, 79:894-898 (2000)

17. Jacob Geroge, Harsh Pal Bais, G. A. Ravishankar. “Optimization of media constituents for shoot regeneration from leaf callus cultures of Decalepis hamiltonii Wight & Arn”, Hort Science 35: 296-299 (2000).

18. Harsh Pal Bais, G. Sudha, B. Suresh &. G. A. Ravishankar, “Silver nitrate influences in vitro root formation in Decalepis hamiltonii Wight & Arn”. Curr. Sci,, 79: 408-410 (2000)

19. Obul Reddy, P. Giridhar, and G. A. Ravishankar, “The effect of triacontanol on B micropropagation of Capsicum frutescens and Decalepis hamiltonii Wight & Arn., Plant Cell Tissue and Organ Culture”. Current Science 81: 1479-1482 (2001)

20. Obul Reddy, P. Giridhar, and G. A. Ravishankar, “The effect of triacontanol on micropropagation of Capsicum frutescens and Decalepis hamiltonii Wight & Arn”, Plant Cell Tissue and Organ Culture, 71: 253-258 (2002)

1. A simple and efficient method for producing viable plants by tissue culture from a Decalepis hamiltonii nodal explants and their effective field establishment by using vesicular arbuscular mycorrhizae (VAM) for effective growth and desired yield of flavour enhanced tubers, said method comprising the steps of:
i) Cutting an explant from a Decalepis hamiltonii plant, said explant consisting essentially of the nodal segment containing axillary bud of said Decalepis plant,
ii) Decontaminating said explant by removing from its surface any contaminant which is potentially harmful to the tissue culture process,
iii) Culturing the decontaminate (ii) explant at a temperature between 25 and 30 degree C., in the presence of cool white light in a first medium which is capable of producing multiple shoots, said first medium having a pH in the range of 5.4 to 6.0 being sterile as a result of autoclaving and comprising:
a) salts
b) vitamins
c) a carbon source
d) phytohormones comprising auxins and cytokinins in a concentration of greater than 4.0 micro molar and
e) a gelling agent
iv) continuing the culture of said explant until proliferating shoots are formed,
v) culturing said shoots in a second medium which is capable of further elongation of shoot at temperature between 25 and 30 degree C. in the presence of a cool white light for at least 4 weeks to generate 6-8 cm long shoots, said second medium having a pH in the range of 5.4 to 6.0, being sterile as a result of autoclaving and comprising:
a) salts
b) vitamins
c) a carbon source
d) phytohormones
e) a gelling agent
vi) Culturing said elongated shoots in a third medium which is capable of inducing roots, at a temperature between 25 and 30 degree C., in the presence of a cool white light for at least 4 weeks to induce rooting, said third medium having a pH in the range of 5.4 to 6.0, being sterile as a result of autoclaving and comprising:
a) salts
b) vitamins
c) a carbon source
d) phytohormones
e) a gelling agent
vii) Hardening the rooted plants by removing carefully from the third medium and washing the medium under running tap water and their subsequent planting in the micropots containing a sand-compost mixture (1:2) under the polythene hoods in the green house for 4 weeks,
viii) Development of seedling based plantlets by sowing the fresh seeds of D. hamilltonii in garden soil. Separation of 12-15 cm long seedlings and planting in pots, inoculation of VAM to the seedling plants and also to hardened micropropagated plants planted in pots comprising:
a) VAM inoculum
b) Mixture of soil: red earth: farm yard manure
ix) Growth of the plants for six months in green house with a photoperiod of 16:8 hourrs relative humidity of 70 to 78 percent during light cycle and 80 to 86 percent during darkness,
x) Measurement of vegetative growth and yield of tubers of the said VAM treated Decalepis hamiltonii platnelts,
xi) Slicing of the said harvested tubers of Decalepis hamiltonii and analysis of the flavor component 2-hydroxy 4 methoxy benzaldehyde by known method using GC.

2. The method according to claim 1 wherein, said first medium, said second medium and said third medium comprise, salts and modified vitamins of Murashige and Skoog medium

3. The method according to claim 2 wherein said nodal segment from a Decalepis plant grown in the field is treated to remove any contaminant

4. The method according to claim 1 wherein, said first medium, said second medium and said third medium comprise salts of Murashige and Skoog medium

5. The method according to claim 1 wherein said first medium, said second medium, and said third medium comprise the following salts of Murashige and Skoog medium:

| Component | Concentration (mg/L) |
|---|---|
| (a) Salts of Murashige and Skoog medium: | |
| NH.sub.4 NO.sub.3 | 1650 |
| KNO.sub.3 | 1900 |
| MgSO.sub.4 7H.sub.2 O | 180.54 |
| MnSO.sub.4 H.sub.2 O | 16.90 |
| ZnSO.sub.4 7H.sub.2 O | 8.6 |
| CuSO.sub.4 5H.sub.2 O | 0.025 |
| CaCl.sub.2 H.sub.2 O | 332.02 |
| KI | 83 |
| CoCl.sub.2 2H.sub.2 O | 0.025 |
| KH.sub.2 PO.sub.4 | 170 |
| H.sub.3 BO.sub.3 | 6.20 |
| Na.sub.2 MoO.sub.4 2H.sub.2 O | 0.25 |
| Fe Na. EDTA | 36.70 |
| Myoinositol | 100.0 |

6. The method according to claim 5 wherein, the concentration of said salts of Murashige and Skoog medium is at the full level on weight by volume basis.

7. The method according to claim 1 wherein, said vitamins of said first medium, said second medium, and said third medium comprise:

| Component | Concentration (mg/L) |
|---|---|
| Nicotinic acid | 0.5 |
| Pyridoxine HCl | 0.5 |
| Thiamine HCl | 0.1 |
| Glycine | 2.0 |
| Biotin | 0.05 |

8. The method according to claim 1 wherein, said carbon source in first medium, said second medium, and said third medium is selected from the group consisting of sucrose and glucose.

9. The method according to claim 1 wherein said carbon source in said first medium, second medium and third medium is at a range of 2-4 percent w/v.

10. The method according to claim 1 wherein said first medium further comprises or cytokinin or combination thereof.

11. The method according to claim 10 wherein the cytokinin is selected from the group consisting of 6-benzylaminopurine (BAP), kinetin and gamma.gamma.dimethyl allyl amino purine (2iP) at a concentration range varying between 4-15 mu. M.

12. The method according to claim 1 wherein the phytohormones in said second medium are selected from the group consisting of cytokinins, auxins and combinations thereof.

13. The method according to claim 10 wherein the auxin in said first medium and said second medium is selected from the group consisting of auxins, indole aetic acid, indole butyric acid and naphthalene acetic acid at a concentration in the range of 0.05 to 10 mu.M.

14. The method according to claim 1 wherein the phytohormones in said first medium and said second medium are cytokinins selected from the group consisting of 6-benzylaminopurine (BAP), kinetin and gamma.gamma.dimethylallyl amino purine (2iP) at a concentration range varying between 4-15 mu. M.

15. The method according to claim 1 wherein the cytokinin in said first medium is selected from the group consisting of 6-benzylaminopurine (BAP), kinetin and gamma.gamma.dimethylallyl amino purine (2iP) at a concentration range varying between 4-15 mu. M.

16. The method according to claim 1 wherein the phytohormone in said second medium is an auxin selected from the group consisting of auxins indole aetic acid, indole butyric acid and naphthalene acetic acid at a concentration in the range of 0.05 to 10 mu.M.

17. The method according to claim 12 wherein said auxin in third medium is selected from the group consisting of auxins indole aetic acid, indole butyric acid and naphthalene acetic acid at a concentration in the range of 0.05 to 10 mu.M.

18. The method according to claim 1 wherein, the explant is decontaminated by dipping in a solution containing at least one sterilizing agent.

19. The method according to claim 18 wherein, said sterilizing agent is selected from the group consisting of sodium hypochlorite, mercuric chloride and ethyl alcohol.

20. The method according to claim 1 wherein the gelling agent is selected from the group consisting of agar and gelrite at a concentration range 0.2 to 0.8% w/v.

21. The method according to claim 1 wherein said shoots can be used for micropropagation of Decalepis plants.

22. The method according to claim 1 wherein the VAM inoculum is added to the soil mixture in pots containing plants in the range of 35 to 70 gm per pot (5700 cc volume soil per pot)

23. The method according to claim 1 wherein the VAM treated plants of Decalepis hamiltonii showed better growth and yield of tubers.

24. The method according to claim 1 wherein the tubers of VAM treated plants of Decalepis hamiltonii showed altered levels of 2 hydroxy 4 methoxy benzaldehyde depending upon the VAM strain used in soil.

25. The method according to claim 1 wherein the Decalepis plant tubers with altered levels of flavor content useful for industrial applications.

26. The method according to claim 1 wherein, the tissue cultured Decalepis hamiltonii plants are useful for effective commercial propagation.

27. The method as claimed in claim 1, wherein the shoot length increases by about 4.5 times.

28. The method as claimed in claim 27, wherein the number of nodes increases by about 1.6 times.

29. The method as claimed in claim 1, wherein the number of leaves increases by about 1.6 times.

30. The method as claimed in claim 1, wherein the total chlorophyll content increases by about 80%.

31. The method as claimed in claim 1, wherein the total number of tubers increases by tuber diameter increases by about 1.6 times.

32. The method as claimed in claim 1, wherein the tuber length increases by about one time.

33. The method as claimed in claim 1, wherein fresh weight of tuber increases by about 1.2 times.

34. The method as claimed in claim 1, wherein the flavour content increases about 4.6 times.

35. A first medium for the efficient production of viable plants by tissue culture from a Decalepis hamiltonii nodal explants and their effective field establishment by using vesicular arbuscular mycorrhizae (VAM) for effective growth and yield of flavour enhanced tubers, said first medium comprising:

| | |
|---|---|
| | Concentration (mg/L) approx. |
| Component | (about) |

A. Salts of MS medium:

| | |
|---|---|
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100 |
| B. Vitamins | |
| Nicotinic acid | 0.5 |
| Pyridoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Cytokinins | 4 to 15 .mu.M |
| Auxins | 0.05 to 10 .mu.M |
| E. Gelling Agents | 0.2 to 0.8% w/v |

36. A second medium for the efficient production of viable plants by tissue culture from a Decalepis hamiltonii nodal explants and their effective field establishment by using vesicular arbuscular mycorrhizae (VAM) for effective growth and yield of flavour enhanced tubers, said first medium comprising:

| | Concentration (mg/L) |
|---|---|
| Component | approx. (about) |
| A. Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100 |
| B. Vitamins | |
| Nicotinic acid | 0.5 |
| Pyridoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Cytokinins | 4 to 15 .mu.M |
| Auxins | 0.05 to 10 .mu.M |
| Gibberellins | 0.2 to 0.4 .mu.M |
| E. Gelling Agents | 0.2 to 0.8% w/v |

37. A third medium for the efficient production of viable plants by tissue culture from a Decalepis hamiltonii nodal explants and their effective field establishment by using vesicular arbuscular mycorrhizae (VAM) for effective growth and yield of flavour enhanced tubers, said first medium comprising:

| | Concentration (mg/L) |
|---|---|
| Component | about (approx.) . . . |
| A. Salts of MS medium: | |
| NH.sub.4 NO.sub.3 | 1650 |
| KNO.sub.3 | 1900 |
| MgSO.sub.4.7H.sub.2 O | 180.54 |
| MgSO.sub.4 H.sub.2 O | 16.90 |
| ZnSO.sub.4.7H.sub.2 O | 8.6 |
| CuSO.sub.4.5H.sub.2 O | 0.025 |
| CaCl.sub.2.2H.sub.2 O | 332.02 |
| KI | 83 |
| CoCl.sub.2 2H.sub.2 O | 0.025 |
| KH.sub.2 PO.sub.4 | 170 |
| H.sub.3 B.sub.3 | 62 |
| Na.sub.2 MoO.sub.4.2H.sub.2 O | 0.25 |
| Fe Na. EDTA | 36.70 |
| Myoinosltol | 100.0 |
| B. Vitamins | |
| Nicotinic acid | 0.5 |
| Pyndoxine HCl | 0.5 |
| Thiamine HCl | 0.1 |
| Glycine | 2.0 |
| Biotin | 0.05 |
| C. Carbon source: | |
| Sucrose/Glucose | 30000.0 |
| D. Hormones (growth regulators) | |
| Auxins | 0.05 to 10 .mu.M |
| E. Gelling Agents | 0.2 to 0.8% w/v |

38. A method of altering the level of flavour in plant Decalepis hamiltonii, said method comprising the tissue culturing the stem explants by the method of claim 1.
